(19) United States
(12) Patent Application Publication
(10) Pub. No.: US 2013/0137662 A1
(43) Pub. Date: May 30, 2013

(54) USE OF CANTHAXANTHIN AND/OR 25-OH D3 FOR IMPROVED REPRODUCTIVITY AND PERFORMANCE OF ROOSTERS

(71) Applicant: Jose Maria Hernandez, Tortuguitas (AR)

(72) Inventor: Jose Maria Hernandez, Tortuguitas (AR)

(21) Appl. No.: 13/518,710

(22) PCT Filed: Dec. 7, 2010
(86) PCT No.: PCT/EP2010/069064

§ 371 Date: Dec. 28, 2012

(30) Foreign Application Priority Data
Dec. 22, 2009 (EP) 09180460.9

(51) Int. Cl. A61K 31/122; A61K 31/593

(52) U.S. Cl. CPC A61K 31/122; A61K 31/593
USPC 514/167; 514/691; 568/367; 552/653

(57) ABSTRACT

The present invention relates to the use canthaxanthin and/or 25-hydroxy vitamin D3 (25-OH D3) for improving reproductive performance of roosters. More particularly, the invention relates to the use of Canthaxanthin and/or 25-hydroxy canthaxanthin in the manufacture of a food or veterinary composition for improving reproductive performance of roosters.

### FIELD OF THE INVENTION

The present invention relates to the use of canthaxanthin and/or at least one vitamin D metabolite, preferably 25-hydroxy vitamin D3 (25-OH D3), for improved reproduction and performance of roosters. More particularly the invention relates to the use of canthaxanthin and/or 25-hydroxy vitamin D3 in the manufacture of a feed or veterinary composition for improving reproduction and performance of roosters.

### BACKGROUND

To maximize the reproduction rate and performance of roosters, optimal nutritional status of animals is essential.

In accordance with the present invention it has been found that problems in chicken reproduction can be eliminated or substantially ameliorated by administering to the roosters an effective amount of Canthaxanthin or 25-OH-D3, optionally a combination of both nutrients.

Tritsch et al. (US 2003/0170324) disclose a feed premix composition of at least 25-OH D3 in an amount between 5% and 50% (wt/wt) dissolved in oil and an antioxidant, an agent encapsulating droplets of 25-OH D3 and oil, and a nutritional additive (e.g., Vitamin D3). The premix may be added to poultry, swine, canine, or feline food. This composition stabilizes 25-OH D3 against oxidation.

Simoes-Nunes et al. (US 2005/0064018) discloses adding a combination of 25-OH Vitamin D3 and Vitamin D3 to animal feed. In particular, about 10 μg/kg to about 100 μg/kg of 25-OH Vitamin D3 and about 200 IU/kg to about 4,000 IU/kg of Vitamin D3 are added to swine feed. This addition improves the pig's bone strength.

Stark et al. (U.S. Pat. No. 5,695,794) disclose adding a combination of 25-OH Vitamin D3 and Vitamin D3 to poultry feed to ameliorate the effects of tibial dyschondroplasia.

Borenstein et al U.S. Pat. No. 5,043,170 discloses the combination of Vitamin D3 and either 1-alpha-hydroxycholecalciferol or 1alpha, 25-dihydroxycholecalciferol to improve egg strength and leg strength in laying hens and older hens.

Fleshner-Barak (WO 03/007916) discloses administration of bisphosphonate compound and natural vitamin D derivative such as 1,25-dihydroxyvitamin D3 or 24,25-dihydroxyvitamin D3, or 25-OH vitamin D3.

Daifotis et al. (WO 03/086415) disclose inhibiting bone resorption by a combination of at least one bisphosphonate compound and from about 100 IU to about 60,000 IU of a no activated metabolite of vitamin D2 and/or vitamin D3.

The aforementioned documents did not teach or suggest that the use of canthaxanthin and 25-OH D3 or a combination thereof would be surprisingly beneficial to improve reproduction and performance of roosters.

### DETAILED DESCRIPTION OF THE INVENTION

As used throughout the specification and claims, the following definitions apply:

“Vitamin D metabolite” means any metabolite of Vitamin D as for example 25-hydroxy vitamin D3, 1,25-dihydroxy vitamin D3 or 24,25-dihydroxy vitamin D3.

“25-OH D3” refers specifically to 25-hydroxy vitamin D3.

“Rooster”, also called a cock or chanticleer, is a male chicken which is meant to include turkeys and ducks.

Canthaxanthin and 25-OH D3 may be obtained from any source, and a composition thereof may be prepared using convenient technology.

In a first aspect, one or more feed compositions suitable for poultry use are provided to administer canthaxanthin or 25-OH D3 and combinations thereof as nutrients to improve hatchability, fertility and lower embryo mortality during the first phase of embryo development.

In a second aspect, a poultry feed is provided which comprises from about 10 μg/kg to about 100 μg/kg of 25-OH D3 and/or from about 2 to 100 ppm canthaxanthin, preferably 2 to 10 ppm.

In another aspect, a method of administering canthaxanthin and/or 25-OH D3 to poultry breeders is provided to improve hatchability, fertility and lower embryo mortality during the first phase of embryo development.

The method for improving hatchability in poultry comprises administering to the animal in need of such treatment an amount of about 2 ppm to 100 ppm of canthaxanthin, preferably 2 to 10 ppm, and/or about 10 μg/kg to about 100 μg/kg of 25-OH D3.

In another aspect, a premix composition for poultry feed comprising 25-hydroxy vitamin D3 and canthaxanthin is provided.

Canthaxanthin and 25-hydroxy vitamin D3 are suitably administered together with the food. The term food as used herein comprises both solid and liquid food as well as drinking fluids such as drinking water. Particularly, inventive ingredients can be added as a formulated powder to a premix containing other minerals, vitamins, amino acids and trace elements which is added to regular animal food and thorough mixing to achieve even distribution therein.

In the manufacture of poultry feed in accordance with the invention, from about 2 ppm to 100 ppm, preferably 2-10 ppm of canthaxanthin and, if required, from about 10 μ/kg to about 100 μg/kg of 25-hydroxy vitamin D3 are added to regular poultry food. Alternatively, a food premix may be prepared on the basis of regular food components by adding these active ingredients to such food components in higher concentration.

According to the present invention the canthaxanthin compound is available under the Trademark ROVIMIX® Hy-D® 1.25% and canthaxanthin under the Trademark CAROPHYLL®Red.

According to the present invention it is further advantageous if the composition also contains one or more of the following ingredients: Vitamin A, Vitamin E, Biotin, copper (e.g. as CuSO₄), zinc (e.g. as ZnSO₄), cobalt (e.g. as CoSO₄), selenium (e.g. as Na₂SeO₃), iodine (e.g. as KI), manganese (e.g. as MnSO₄) and/or calcium (e.g. as CaSO₄).

The following non-limiting Examples are presented to better illustrate the invention.

### EXAMPLE 1

### Effect of Carophyll Red (Canthaxanthin) on the Productive and Reproductive Performance of Roosters

Facilities and Equipment

The trial was conducted at the experimental laying house, measuring 210 m², using 40 cages for male breeders (0.33×0.60×0.60 m). This open-type house is equipped with side curtains, and metal roofing. Each cage is fitted with a cup drinker and through feeder.

Animals

Forty pre-selected 40-week old White Plymouth Rock males were used in the trial, and housed individually in cages.

Management

The period between the 37^{th} and 39^{th} weeks of age was considered the pre-experimental stage, and included the selection of roosters to be used in the trial. The selection was based on phenotypic assessment, response to abdominal massage for ejaculation stimulation, and semen volume of the ejaculate.

The trial was conducted between weeks 40 and 59 of age, and the experimental period was divided in 5 study periods for body weight and feed consumption evaluation, as follows: period I—40 to 43 weeks of age, period II—44 to 47 weeks of age, period III—48 a 51 weeks of age, period IV—52 to 55 weeks of age, and period V—56 to 59 weeks of age.

The roosters were individually weighed every 28 days for body weight measurements. The amount of feed supplied and the leftovers were also weighed for feed consumption calculation. The average body weight at start of the trial was 2,936 grams. During the experimental period, the roosters received the treatments described in Table 1, and were fed ad libitum, with feed being supplied every day during early morning.

The diets fed during the trial were formulated according to the standard LAVIC feeds for roosters, with or without the addition of the tested product, formulated to meet the requirements according to the life stage of the birds. The diets contained only plant products, and were based on corn and soybean meal.

In 15 day-intervals, semen was collected after 1 p.m. using the abdominal massage method. The rooster was held by the legs, and the breast touched a soft surface on the cage. The obtained semen sample was analyzed for motility, morphological changes, and sperm concentration.

For the assessment of motility and vigor, the fresh semen sample was placed on a slide, covered by a glass slip, and analyzed under a light microscope, at 40× magnification. During the evaluation, the sample was maintained on a heated plate at 40° C. Motility was assessed by comparing the percentage (%) of mobile and immobile spermatozoa, and recording of the percentage of mobile sperm. Vigor was assessed according to a 0 to 5 score scale, being score 0 representative of complete sperm immobility, and score 5 indicative of intense, vigorous, and progressive movement, with wave formation.

For the evaluation of sperm morphology and concentration, samples of the ejaculate were diluted in formalin citrate solution in Eppendorf tubes. For measuring sperm concentration, a 10 μl semen sample was added to 1 ml of formalin citrate solution, and sperm cells were counted in a Neubauer counting chamber following a diagonal line, and the result expressed in number of cells per mm³ of semen. For the final analysis, the results were expressed in number of cells/ml.

For the assessment of morphological anomalies of sperm cells, a 10 μl semen sample was added to 1 ml of formalin citrate solution, and evaluated using a phase contrast microscope, at a 1000× magnification. One hundred (100) cells were evaluated, and the morphological changes were expressed in percentages.

Treatments

Table 1 describes the experimental treatments.

TABLE 1
Experimental treatments used in the trial conducted from August
to December 2008, with Plymouth Rock White roosters.

| Treatments | Carophyll Red (ppm) |
|---|---|
| 1 | 0 |
| 2 | 60 |

Experimental Design

The experimental design was completely randomized, with two treatments and 20 repetitions each, where each bird was considered a repetition.

Results

TABLE 2
Final body weights (grams) of White Plymouth Rock roosters
for each trial period, and averages of all periods.

| | Week | | | | | |
|---|---|---|---|---|---|---|
| Treatments | 43rd | 47th | 51st | 55th | 59th | Average |
| Control | 2900 | 2953 | 2895 | 2991 | 3082 | 2952 |
| Carophyll Red | 2967 | 3003 | 2984 | 3058 | 3073 | 3008 |
| P | 0.2139 | 0.3794 | 0.1531 | 0.1611 | 0.9956 | 0.2613 |
| CV | 0.74 | 0.80 | 0.83 | 0.63 | 0.98 | 0.68 |

TABLE 3
Total feed consumption (grams) of White Plymouth Rock roosters
for each period and total average feed consumption

| | Periods | | | | | |
|---|---|---|---|---|---|---|
| Treatments | I | II | III | IV | V | Average |
| Control | 3005 | 2956 | 2981 b | 3002 b | 3021 | 2998 |
| Carophyll Red | 3071 | 3090 | 3170 a | 3263 a | 3059 | 3132 |
| P | 0.3168 | 0.1100 | 0.0697 | 0.0223 | 0.9004 | 0.1191 |
| CV | 0.97 | 1.00 | 1.24 | 1.29 | 1.64 | 1.02 |

a>b (P < 0.1)—Duncan's test

TABLE 4
Daily feed consumption (grams/bird/day) of White
Plymouth Rock roosters by experimental period

| | Periods | | | | |
|---|---|---|---|---|---|
| Treatments | I | II | III | IV | V |
| Control | 107 | 105 | 106 b | 107 b | 108 |
| Carophyll Red | 110 | 110 | 113 a | 116 a | 109 |
| P | 0.3168 | 0.1100 | 0.0697 | 0.0223 | 0.9004 |
| CV | 0.97 | 1.00 | 1.24 | 1.29 | 1.64 |

a>b (P < 0.1)—Duncan's test

TABLE 5
Morphological changes of spermatozoa (%) of roosters
in periods I to III (two collections)

| | Periods | | | | | |
|---|---|---|---|---|---|---|
| | I | | II | | III | |
| Treatments | 1° | 2° | 1° | 2° | 1° | 2° |
| Control | 25.6 | 28.3 a | 23.7 a | 13.1 | 18.2 a | 24.3 a |
| Carophyll Red | 28.2 | 24.4 b | 19.2 b | 10.3 | 16.1 b | 18.3 b |
| P | 0.4217 | 0.0006 | 0.0006 | 0.4644 | 0.0100 | 0.0001 |
| CV | 6.31 | 3.78 | 5.75 | 5.80 | 4.81 | 6.09 |

a>b (P < 0.1)—Duncan's test

TABLE 6
Morphological changes of spermatozoa (%) of roosters in periods
IV and V (two collections) and averages for all periods

| | Periods | | | | |
|---|---|---|---|---|---|
| Treat- | IV | | V | | I-V |
| ments | 1st | 2nd | 1st | 2nd | Average |
| Control | 19.3 a | 21.4 | 19.4 a | 25.6 | 21.9 a |
| Carophyll Red | 16.1 b | 19.9 | 17.2 b | 24.6 | 19.6 b |
| P | 0.0001 | 0.1499 | 0.0631 | 0.1855 | 0.0001 |
| CV | 4.46 | 4.94 | 7.71 | 3.26 | 1.89 |

a>b (P < 0.1)—Duncan's test

TABLE 7
Sperm motility (%) of the roosters in
periods I to III (two collections)

| | Periods | | | | | |
|---|---|---|---|---|---|---|
| | I | | II | | III | |
| Treatments | 1st | 2nd | 1st | 2nd | 1st | 2nd |
| Control | 90.25 | 91.00 | 90.00 | 89.00 | 91.75 | 91.25 |
| Carophyll Red | 92.50 | 92.00 | 90.25 | 91.50 | 92.50 | 92.50 |
| P | 0.9110 | 0.3502 | 0.8780 | 0.1251 | 0.4706 | 0.1492 |
| CV | 2.29 | 1.79 | 2.63 | 2.80 | 1.75 | 1.45 |

TABLE 8
Sperm motility (%) of the roosters in periods IV and
V (two collections) and averages for all periods

| | Periods | | | | |
|---|---|---|---|---|---|
| Treat- | IV | | V | | I-V |
| ments | 1st | 2nd | 1st | 2nd | Average |
| Control | 92.50 | 91.00 | 92.50 | 91.50 | 91.40 b |
| Carophyll Red | 93.00 | 91.25 | 92.75 | 93.75 | 92.50 a |
| P | 0.5372 | 0.7954 | 0.7950 | 0.2811 | 0.0119 |
| CV | 1.37 | 1.60 | 1.77 | 3.89 | 0.72 |

a>b (P < 0.1)—Duncan's test

TABLE 9
Sperm vigor score of roosters in periods
I to III period (two collections)

| | Periods | | | | | |
|---|---|---|---|---|---|---|
| | I | | II | | III | |
| Treatments | 1st | 2nd | 1st | 2nd | 1st | 2nd |
| Control | 4.71 | 4.35 | 4.50 | 4.38 | 4.28 | 4.35 |
| Carophyll Red | 4.65 | 4.47 | 4.57 | 4.65 | 4.61 | 4.61 |
| P | 0.7239 | 0.6313 | 0.7679 | 0.1994 | 0.1900 | 0.1133 |
| CV | 5.26 | 7.92 | 7.26 | 7.04 | 8.59 | 5.53 |

TABLE 10
Sperm vigor score of roosters in periods IV and
V (two collections) and averages for all periods

| | Periods | | | | |
|---|---|---|---|---|---|
| Treat- | IV | | V | | I-V |
| ments | 1st | 2nd | 1st | 2nd | Average |
| Control | 4.32 | 4.24 | 4.38 | 4.37 | 4.38 b |
| Carophyll Red | 4.53 | 4.31 | 4.61 | 4.53 | 4.56 a |
| P | 0.3237 | 0.7158 | 0.2530 | 0.6271 | 0.0312 |
| CV | 8.16 | 7.57 | 7.06 | 9.55 | 2.84 |

a>b (P < 0.1)—Duncan's test

TABLE 11
Sperm concentration (number of cells ×
10⁸) of roosters for periods I to III (two collections)

| | Periods | | | | | |
|---|---|---|---|---|---|---|
| | I | | II | | III | |
| Treatments | 1st | 2nd | 1st | 2nd | 1st | 2nd |
| Control | 4.69 | 4.00 | 4.67 | 3.94 b | 4.61 b | 5.79 |
| Carophyll Red | 4.82 | 4.31 | 5.06 | 5.05 a | 5.02 a | 5.83 |
| P | 0.5950 | 0.2795 | 0.4657 | 0.0001 | 0.0588 | 0.5485 |
| CV | 5.01 | 4.48 | 3.78 | 3.25 | 2.95 | 3.11 |

a>b (P < 0.1)—Duncan's test

TABLE 12
Sperm concentration (number of cells × 10⁸) of roosters
for periods IV and V (two collections) and average for all periods

| | Periods | | | | |
|---|---|---|---|---|---|
| Treat- | IV | | V | | I-V |
| ments | 1st | 2nd | 1st | 2nd | Average |
| Control | 3.00 b | 5.99 b | 4.61 b | 2.65 | 4.40 b |
| Carophyll Red | 3.84 a | 6.46 a | 5.30 a | 2.84 | 4.85 a |
| P | 0.0001 | 0.0084 | 0.0056 | 0.1152 | 0.0002 |
| CV | 2.56 | 1.46 | 2.70 | 2.59 | 1.24 |

a>b (P < 0.1)—Duncan's test

Conclusion

The addition of Carophyll Red to the diets resulted in significant improvements in sperm concentration and vigor, and reduced the incidence of morphological changes seen in spermatozoa produced by White Plymouth Rock roosters during the experimental period, from 40 to 59 weeks of age.

### EXAMPLE 2

### Effect of Carophyll Red and 25-OH-D3 on the Reproductive Performance of Roosters

The trial of example 2 was conducted as described for example 1.

Treatments

Table 13 describes the four (4) treatments used in this trial.

TABLE 13
Experimental treatments used in the trial conducted from August
to December 2008, with Plymouth Rock White roosters.

| Treatments | Carophyll Red (ppm) | HY-D (ppb) |
|---|---|---|
| 1 | 0 | 0 |
| 2 | 60 | 0 |
| 3 | 0 | 69 |
| 4 | 60 | 69 |

Results

TABLE 14
Final body weights (grams) of White Plymouth Rock roosters
for each trial period, and averages of all periods.

| | Week of Age | | | | | |
|---|---|---|---|---|---|---|
| Treatments | 43 | 47 | 51 | 55 | 59 | Average |
| Control | 2900 | 2953 | 2895 ab | 2991 ab | 3082 | 2952 |
| Carophyll | 2967 | 3003 | 2984 a | 3058 a | 3073 | 3008 |
| HyD | 2850 | 2870 | 2849 b | 2946 b | 3040 | 2911 |
| Carophyll + | 2933 | 2948 | 3018 a | 3079 a | 3119 | 3012 |
| HyD | | | | | | |
| P | 0.2148 | 0.2497 | 0.0310 | 0.0517 | 0.7460 | 0.2136 |
| CV | 0.79 | 0.93 | 0.85 | 0.69 | 0.96 | 0.75 |

a>b (P < 0.1)—Duncan's test

TABLE 15
Total feed consumption (grams) of White Plymouth Rock roosters
for each period and total average feed consumption

| | Periods | | | | | |
|---|---|---|---|---|---|---|
| Treatments | I | II | III | IV | V | Average |
| Control | 3005 | 2956 ab | 2981 b | 3002 b | 3021 | 2998 ab |
| Carophyll | 3071 | 3090 a | 3170 ab | 3263 a | 3059 | 3132 ab |
| HyD | 2937 | 2884 b | 2988 b | 3058 ab | 3009 | 2975 b |
| Carophyll + | 3103 | 3062 a | 3229 a | 3213 ab | 3148 | 3151 a |
| HyD | | | | | | |
| P | 0.1399 | 0.0659 | 0.0324 | 0.0519 | 0.6309 | 0.0704 |
| CV | 1.03 | 1.11 | 1.28 | 1.30 | 1.62 | 1.03 |

a>b (P < 0.1)—Duncan's test

TABLE 16
Daily feed consumption (grams/bird/day) of White
Plymouth Rock roosters by experimental period

| | Periods | | | | |
|---|---|---|---|---|---|
| Treatments | I | II | III | IV | V |
| Control | 107 | 105 ab | 106 b | 107 b | 108 |
| Carophyll | 110 | 110 a | 113 ab | 116 a | 109 |
| HyD | 105 | 103 b | 106 b | 109 ab | 107 |
| Carophyll + HyD | 111 | 109 a | 115 a | 114 ab | 112 |
| P | 0.1399 | 0.0659 | 0.0324 | 0.0519 | 0.6309 |
| CV | 1.76 | 1.91 | 2.18 | 2.21 | 2.77 |

a>b (P < 0.1)—Duncan's test

TABLE 17
Morphological changes of spermatozoa (%) of roosters
in periods I to III (two collections)

| | Periods | | | | | |
|---|---|---|---|---|---|---|
| | I | | II | | III | |
| Treatments | 1st | 2nd | 1st | 2nd | 1st | 2nd |
| Control | 25.6 | 28.3 a | 23.7 a | 13.1 a | 18.2 a | 24.3 a |
| Carophyll | 28.2 | 24.4 b | 19.2 b | 10.3 a | 16.1 b | 18.3 b |
| HyD | 24.9 | 20.5 c | 17.2 c | 12.5 b | 12.0 c | 16.3 c |
| Carophyll + | 25.1 | 19.1 c | 16.4 c | 12.6 a | 13.0 c | 17.5 bc |
| HyD | | | | | | |
| P | 0.3841 | 0.0001 | 0.0001 | 0.0001 | 0.0001 | 0.0001 |
| CV | 4.78 | 3.60 | 6.91 | 5.37 | 5.87 | 5.52 |

a>b>c (P < 0.1)—Duncan's test

TABLE 18
Morphological changes of spermatozoa (%) of roosters in periods
IV and V (two collections) and averages for all periods

| | Periods | | | | |
|---|---|---|---|---|---|
| Treat- | IV | | V | | I-V |
| ments | 1st | 2nd | 1st | 2nd | Average |
| Control | 19.3 a | 21.4 a | 19.4 a | 25.6 a | 21.9 a |
| Carophyll | 16.1 b | 19.9 ab | 17.2 b | 24.6 a | 19.6 b |
| HyD | 13.8 c | 18.8 b | 14.3 c | 12.5 b | 16.1 d |
| Carop + | 15.0 bc | 18.7 b | 15.6 bc | 13.7 c | 16.7 c |
| HyD | | | | | |
| P | 0.0001 | 0.0001 | 0.0001 | 0.0001 | 0.0001 |
| CV | 4.53 | 4.21 | 6.67 | 4.01 | 1.84 |

a>b>c>d (P < 0.1)—Duncan's test

TABLE 19
Sperm motility (%) of the roosters in periods I to III (two collections)

| | Períodos | | | | | |
|---|---|---|---|---|---|---|
| | I | | II | | III | |
| Treatments | 1st | 2nd | 1st | 2nd | 1st | 2nd |
| Control | 90.25 | 91.00 b | 90.00 | 89.00 b | 91.75 | 91.25 b |
| Carophyll | 92.50 | 92.00 ab | 90.25 | 91.50 ab | 92.50 | 92.50 ab |
| HyD | 90.75 | 93.25 a | 91.50 | 92.25 a | 92.75 | 93.50 a |
| Carophyll + | 89.25 | 93.50 a | 91.25 | 92.00 a | 93.25 | 93.25 a |
| HyD | | | | | | |
| P | 0.2106 | 0.0459 | 0.7272 | 0.0552 | 0.4211 | 0.0587 |
| CV | 2.73 | 1.68 | 2.72 | 2.29 | 1.56 | 1.51 |

a>b (P < 0.1)—Duncan's test

TABLE 20
Sperm motility (%) of the roosters in periods IV and
V (two collections) and averages for all periods

| | Periods | | | | |
|---|---|---|---|---|---|
| Treat- | IV | | V | | I-V |
| ments | 1st | 2nd | 1st | 2nd | Average |
| Control | 92.5 b | 91.00 | 92.50 | 91.50 | 91.40 b |
| Carophyll | 93.00 ab | 91.25 | 92.75 | 93.75 | 92.50 a |
| HyD | 94.25 a | 91.25 | 93.50 | 94.25 | 93.05 a |
| Carophyll + HyD | 93.75 ab | 91.25 | 93.25 | 94.25 | 92.80 a |
| P | 0.0859 | 0.9902 | 0.6979 | 0.2258 | 0.0003 |
| CV | 1.24 | 1.55 | 1.61 | 2.83 | 0.66 |

a>b (P < 0.1)—Duncan's test

TABLE 21
Sperm vigor score of roosters in periods
I to III period (two collections)

| | Periods | | | | | |
|---|---|---|---|---|---|---|
| | I | | II | | III | |
| Treatments | 1st | 2nd | 1st | 2nd | 1st | 2nd |
| Control | 4.71 | 4.35 | 4.50 | 4.38 | 4.28 | 4.35 |
| Carophyll | 4.65 | 4.47 | 4.57 | 4.65 | 4.61 | 4.61 |
| HyD | 4.40 | 4.75 | 4.59 | 4.65 | 4.68 | 4.40 |
| Carophyll + | 5.00 | 4.00 | 5.00 | 5.00 | 4.00 | 5.00 |
| HyD | | | | | | |
| P | 0.3723 | 0.2390 | 0.8532 | 0.3748 | 0.2624 | 0.2724 |
| CV | 6.46 | 7.00 | 6.72 | 6.52 | 7.94 | 5.58 |

TABLE 22
Sperm vigor score of roosters in periods IV and
V (two collections) and averages for all periods

| | Periods | | | | |
|---|---|---|---|---|---|
| Treat- | IV | | V | | I-V |
| ments | 1st | 2nd | 1st | 2nd | Average |
| Control | 4.32 | 4.24 | 4.38 | 4.37 | 4.38 b |
| Carophyll | 4.53 | 4.31 | 4.61 | 4.53 | 4.56 a |
| HyD | 4.53 | 4.21 | 4.44 | 4.40 | 4.55 a |
| Carophyll + HyD | 5.00 | 4.00 | 5.00 | 4.00 | 4.57 a |
| P | 0.5588 | 0.9403 | 0.5933 | 0.9207 | 0.0827 |
| CV | 7.77 | 7.16 | 7.49 | 9.48 | 2.94 |

a>b (P < 0.1)—Duncan's test

TABLE 23
Sperm concentration (number of cells ×
10⁸) of roosters for periods I to III (two collections)

| | Periods | | | | | |
|---|---|---|---|---|---|---|
| | I | | II | | III | |
| Treatments | 1st | 2nd | 1st | 2nd | 1st | 2nd |
| Control | 4.69 | 4.00 c | 4.67 c | 3.94 c | 4.61 c | 5.79 b |
| Carophyll | 4.82 | 4.31 bc | 5.06 bc | 5.05 b | 5.02 b | 5.83 b |
| HyD | 4.43 | 4.73 ab | 5.51 ab | 5.43 b | 5.65 a | 7.52 a |
| Carophyll + | 4.65 | 5.02 a | 6.29 a | 6.19 a | 6.17 a | 7.60 a |
| HyD | | | | | | |
| P | 0.7855 | 0.0034 | 0.0007 | 0.0001 | 0.0001 | 0.0001 |
| CV | 4.37 | 3.71 | 3.56 | 2.56 | 2.57 | 2.46 |

a>b>c (P < 0.1)—Duncan's test

TABLE 24
Sperm concentration (number of cells × 10⁸) of roosters
for periods IV and V (two collections) and average for all periods

| | Periods | | | | |
|---|---|---|---|---|---|
| Treat- | IV | | V | | I-V |
| ments | 1st | 2nd | 1st | 2nd | Average |
| Control | 3.00 c | 5.99 c | 4.61 d | 2.65 b | 4.40 d |
| Carophyll | 3.84 b | 6.46 b | 5.30 c | 2.84 b | 4.85 c |
| HyD | 4.49 a | 6.42 b | 6.20 b | 3.53 a | 5.40 b |
| Carophyll + HyD | 4.75 a | 7.50 a | 7.22 a | 3.66 a | 5.91 a |
| P | 0.0001 | 0.0001 | 0.0001 | 0.0001 | 0.0001 |
| CV | 2.09 | 1.45 | 2.18 | 3.56 | 1.04 |

a>b>c>d (P < 0.1)—Duncan's test

### CONCLUSIONS

The addition of HyD or Carophyll Red to the diets resulted in significant improvements in sperm concentration, and reduced the incidence of morphological changes seen in spermatozoa produced by White Plymouth Rock roosters during the experimental period, from 40 to 59 weeks of age.

1. The use of canthaxanthin and/or at least one vitamin D metabolite for improving reproductive performance of roosters.

2. The use according to claim 1, wherein the vitamin D metabolite is 25-hydroxy vitamin D3.

3. The use of canthaxanthin and or 25-hydroxy vitamin D3 in the manufacture of a food or veterinary composition for improving reproductive performance of roosters.

4. The use as in claim 1 in the manufacture of a poultry food comprising from about 10 μg/kg to about 100 μg/kg of 25-hydroxy vitamin D3 and from about 2 to 100 ppm canthaxanthin, preferably from about 2 ppm to 10 ppm.

5. A method for improving reproductive performance of roosters, which comprises administering to an animal in need of such treatment an amount of about 2 ppm to 100 ppm, preferably 2 to 10 ppm of canthaxanthin and/or about 10 μg/kg to about 100 μg/kg of at least one vitamin D metabolite.

6. The method according to claim 5, wherein the vitamin D metabolite is 25-hydroxy vitamin D3.

7. The method according to claim 6, wherein canthaxanthin and 25-hydroxy vitamin D3 are administered together.

8. A premix composition comprising 25-hydroxy vitamin D3 and canthaxanthin for use in breeder feed to improve hatchability.
